(19) United States
(12) Patent Application Publication
(10) Pub. No.: US 2023/0053659 A1
(43) Pub. Date: Feb. 23, 2023

(54) System for reducing pathogenic bioburden using a UV-C light emitting device and sensors

(71) Applicants: William Hood, Lake Bluff, IL (US); Chase Hood, Barrington, IL (US); Garik Griffin, Lake Forest, IL (US); Brandon Donnelly, Oxford (GB)

(72) Inventors: William Hood, Lake Bluff, IL (US); Chase Hood, Barrington, IL (US); Garik Griffin, Lake Forest, IL (US); Brandon Donnelly, Oxford (GB)

(21) Appl. No.: 17/931,451

(22) Filed: Sep. 12, 2022

Related U.S. Application Data
(63) Continuation-in-part of application No. 17/820,875, filed on Aug. 18, 2022.
(60) Provisional application No. 63/234,345, filed on Aug. 18, 2021.

(51) Int. Cl. A61L 2/10; A61L 2/24

(52) U.S. Cl. CPC A61L 2/10; A61L 2/24; A61L 2202/11; A61L 2202/14

(57) ABSTRACT

In one embodiment, a system for reducing pathogenic bioburden in an environment comprises a light emitting device comprising one or more light sources emitting UV-C light, two or more sensors generating environmental data, and a processor communicatively coupled to the two or more sensors and the light emitting device, the processor performing an analysis on the environmental data from each sensor of the two or more sensors and adjusting the light flux emitted from the light emitting device based at least in part on the environmental data from the two or more sensors. The light flux emitted from the light emitting device may be adjusted based at least on temperature, humidity, and occupancy of the environment sensed by the two or more sensors.

### CROSS REFERENCE TO RELATED APPLICATION

This application in a continuation-in-part application of U.S. application Ser. No. 17/820,875 entitled “Light emitting device emitting UV-C radiation at different wavelengths upward and downward,” filed on Aug. 18, 2022, which claims the benefit of U.S. Provisional Application No. 63/234,345, entitled “Short wave ultraviolet light emitting device,” filed on Aug. 18, 2021, the entire contents of which are incorporated by reference herein.

### BACKGROUND

The subject matter disclosed herein generally relates to light emitting devices such as light fixtures, light bulbs, replacement light bulbs, or devices comprising ultraviolet light emitting devices, systems comprising the UV-C light emitting devices and their components and method of manufacture and controlling light flux output and environmental variables. Light emitting devices are needed that emit light at wavelengths that efficiently and safely inactivate and/or reduce pathogenic bioburden in areas.

### BRIEF DESCRIPTION OF THE DRAWINGS

FIG. 1 is a side view of a light emitting device comprising a UV-C light source comprising one or more first light sources emitting UV-C light downward when the light emitting device is mounted to a ceiling.

FIG. 2 is a bottom view of the light emitting device of FIG. 1.

FIG. 3 is a side view of a light emitting device comprising a first UV-C light source emitting UV-C light downward and a second UV-C light source emitting UV-C light upward.

FIG. 4 is a schematic view of system comprising a plurality of UV-C light emitting devices, remote sensors, a remote processor, a mobile device, and a robot

### DETAILED DESCRIPTION OF THE INVENTION

The features and other details of several embodiments will now be more particularly described. It will be understood that particular embodiments described herein are shown by way of illustration and not as limitations. The principal features can be employed in various embodiments without departing from the scope of any particular embodiment. The present inventive subject matter now will be described more fully hereinafter with reference to the accompanying drawings, in which embodiments of the inventive subject matter are shown. However, this inventive subject matter should not be construed as limited to the embodiments set forth herein. Rather, these embodiments are provided so that this disclosure will be thorough and complete, and will fully convey the scope of the inventive subject matter to those skilled in the art. Like numbers refer to like elements throughout. As used herein the term “and/or” includes any and all combinations of one or more of the associated listed items.

The terminology used herein is for the purpose of describing particular embodiments only and is not intended to be limiting of the inventive subject matter. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

When an element such as a layer, region or substrate is referred to herein as being “on” or extending “onto” another element, it can be directly on or extend directly onto the other element or intervening elements may also be present. In contrast, when an element is referred to herein as being “directly on” or extending “directly onto” another element, there are no intervening elements present. Also, when an element is referred to herein as being “connected” or “coupled” to another element, it can be directly connected or coupled to the other element or intervening elements may be present. In contrast, when an element is referred to herein as being “directly connected” or “directly coupled” to another element, there are no intervening elements present. In addition, a statement that a first element is “on” a second element is synonymous with a statement that the second element is “on” the first element.

Although the terms “first”, “second”, etc. may be used herein to describe various elements, components, regions, layers, sections and/or parameters, these elements, components, regions, layers, sections and/or parameters should not be limited by these terms. These terms are only used to distinguish one element, component, region, layer, section, or property from another region, layer, section, or property. Thus, a first element, component, region, layer, section, or property discussed below could be termed a second element, component, region, layer, section, or property without departing from the teachings of the present inventive subject matter.

Furthermore, relative terms, such as “lower” or “bottom” and “upper” or “top,” may be used herein to describe one element's relationship to another element as illustrated in the Figures or description. Such relative terms are intended to encompass different orientations of the device in addition to the orientation depicted in the Figures. For example, if the device in the Figures is turned over, elements described as being on the “lower” or “bottom” side of other elements would then be oriented on the “upper” or “top” sides of the other elements. The exemplary term “lower”, can therefore, encompass both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below. Likewise, “lower side” and “upper side” can therefore, encompass both an orientation of “lower side” and “upper side,” depending on the particular orientation of the figure.

As used herein “Ultraviolet C” or “UV-C” radiation is radiation within a wavelength range of 100 nanometers to 280 nanometers, “nm” is an abbreviation of nanometers, and “mW” is an abbreviation for milliwatts. “UV-C” referenced herein broadly includes the wavelength range from 100 nanometers to 280 nanometers and embodiments discussing UV-C light include light sources that may emit light with peak wavelengths (such as light with a 222 nm peak wavelength and/or light with a 254 nm peak wavelength) and/or wavelength bandwidths disclosed herein.

As used herein pathogenic bioburden refers to the number of pathogens such as bacterium, protozoan, prion, viroid, or fungus living on a surface.

### Light Emitting Device

In one embodiment, a light emitting device is constructed to emit UV-C radiation downward into a room to inactivate and/or reduce pathogenic bioburden in the environment such as a room. In a further embodiment, the light emitting device emits UV-C radiation upward toward a ceiling. In one embodiment, a light emitting device emits UV-C radiation downward with a first peak wavelength and UV-C radiation upward with a second peak wavelength different than the first wavelength. For example, in one embodiment, a light fixture installed to or within the ceiling emits light with a peak wavelength at 222 nm downward, emits light with a peak wavelength at 254 nm upward, or emits light with a peak wavelength at 222 nm downward and emits light with a peak wavelength of 254 nm upward.

### Light Source

In one embodiment, a light emitting device comprises one or more light sources selected from the group excimer lamp, microcavity microplasma excimer lamp, pulsed xenon lamp, light emitting diode, low pressure mercury lamp (standard output quartz, high output quartz, compact quartz or softglass), UV amalgam lamp, medium pressure UV lamp, low-pressure mercury lamp, gas-discharge lamp, laser, and solid state laser that produces UV-C light and/or light at other wavelengths disclosed herein. In one embodiment, the light source may comprise one or more optical wavelength filters to reduce the light output in one or more wavelength bands (such as visible, UV-A, and/or UV-B bands, for example).

For example, suitable light sources emitting UV-C light (such as 222 nm) include micro-cavity plasma arrays comprising one or more noble gases, one or more halogen gases, or a mixture of at least one halogen gas with one or more noble gases, such as those disclosed in U.S. Pat. No. 11,004,673, and light sources disclosed in US Patent Publication Nos. 20110275272, 20120319559, 20130071297, and International PCT Application Publication No. WO2007011865, the entire contents of each are incorporated by reference herein.

In another embodiment, the one or more light sources include one or more arrays of light sources (such as light emitting diodes or microcavities of plasma) which may be mounted on linear circuit boards or substrates. In one embodiment, the light emitting device comprises one or more linear sections of circuit boards or substrates with discrete UV-C LED packages (which comprise at least one UV-C LED die) and/or microcavities of plasma. In another embodiment, a light emitting device comprises a plurality of light sources within one package disposed to emit light toward a surface for illumination. In one embodiment, the light emitting device comprises at least one selected from the group of: 2, 3, 4, 5, 6, 8, 9, 10, 20, 40, 60, 80, 100, 120, 140, 160, 180, 200, 220, 240, 260, 280, 300, 320, 340, 360, 380, and 400 light sources, light emitting diodes, or microcavities of plasma. In one embodiment, the average dimension, A, of the LEDs in one or more linear arrays of LEDs in a linear direction is less than one selected from the group of 10 mm, 8 mm, 6 mm, 5 mm, 4 mm, 3 mm, and 2 mm. In one embodiment, the light emitting device comprises one or more LEDs of package or case type selected from the group: 0402, 0404, 0603, 0604, 0605, 0606, 0802, 0805, 0806, 0807, 1008, 1050, 1104, 1106, 1204, 1205, 1206, 1208, 1209, 1210, 1305, 1307, 1308, 1411, 1412, 1505, 1608, 1610, 1612, 1616, 1810, 1819, 1908, 1916, 2012, 2024, 2106, 2120, 2122, 2214, 2221, 2220, 2432, 2508, 2520, 2810, 2832, 3010, 3015, 3020, 3022, 3025, 3028, 3034, 3107, 3122, 3228, 3210, 3216, 3224, 3228, 3430, 3519, 3528, 3528, 3632, 4028, 4040, 4234, 4238, 4242, 5050, 5630, 6050, and 7950 where the first two numbers typically represent the length and the second two numbers represent the width in tenths of millimeters. For example, the 5630 LED package type has a length of 5.6 millimeters and a width of 3 millimeters.

### Spectral Properties of the One or More Light Sources

In one embodiment, the light emitting device comprises one or more light sources arranged to emit UV-C light with the same or different first peak wavelength, first center wavelength, first average wavelength, and first wavelength bandwidth upward and/or downward when the light emitting device is mounted in the upper region of a room. In this embodiment, the one or more light sources may have a first peak wavelength, first center wavelength, or first average wavelength selected from the group: 218 nm, 219 nm, 220 nm, 221 nm, 222 nm, 223 nm, 224 nm, 225 nm, 226 nm, 193 nm, 248 nm, 308 nm, 351 nm, and 282 nm. In one embodiment, the one or more light sources may have a first peak wavelength, first center wavelength, or first average wavelength within the wavelength range of 100 nm to 280 nm, 150 nm to 250 nm, 200 nm to 250 nm, 215 nm to 250 nm, 215 nm to 230 nm, 220 nm to 225 nm, and 221 nm and 223 nm. In one embodiment, the one or more light sources emit light with the same or different wavelength bandwidths (full wavelength bandwidth at half maximum intensity) less than, equal to, or greater than one selected from the group: 1 nm, 2 nm, 3 nm, 4 nm, 5 nm, 10 nm, 15 nm, 20 nm, 25 nm, 30 nm, 40 nm, 50 nm, and 70 nm.

In one embodiment, the light emitting device comprises one or more first light sources emitting UV-C light downward to irradiate the environment below the light emitting device (such as air and the floor and other surfaces) and optionally a second light source emitting UV-C light upward to irradiate the environment above the light emitting device (such as air, the ceiling, and other surfaces). In this embodiment, the one or more first light sources may emit light with a first peak wavelength, first center wavelength, or first average wavelength (222 nm for example) and the one or more second light sources may emit light with a second peak wavelength, second center wavelength, or second average wavelength different from the first peak wavelength, first center wavelength, or first average wavelength, respectively, (254 nm for example). In one embodiment, a method of inactivating and/or reducing pathogenic bioburden in an environment comprises emitting first UV-C light (such as 222 nm light for example) from one or more first light sources in a light emitting device downward to irradiate the environment below the light emitting device (such as air and the floor and other surfaces) and optionally emitting UV-C light (that may have a different peak wavelength, center wavelength, or average wavelength than the first UV-C light, (such as 254 nm for example) from one or more second light sources to irradiate the environment above the light emitting device (such as air, the ceiling, and other surfaces).

In some embodiments, the light emitting device comprises only one or more light sources emitting light downward when the light emitting device is positioned in room or mounted to or near the ceiling, for example. In one embodiment, the one or more second light sources may have a second peak wavelength, second center wavelength, or second average wavelength selected from the group: 218 nm, 219 nm, 220 nm, 221 nm, 222 nm, 223 nm, 224 nm, 225 nm, 226 nm, 193 nm, 248 nm, 265 nm, 273 nm, and 280 nm. In one embodiment, the one or more second light sources may have a second peak wavelength, second center wavelength, or second average wavelength within the wavelength range of 100 nm to 280 nm, 150 nm to 250 nm, 200 nm to 250 nm, 215 nm to 250 nm, 215 nm to 230 nm, 250 nm to 260 nm, 252 nm to 257 nm, 220 nm to 225 nm, 221 nm and 223 nm, 230 nm to 240 nm, 240 nm to 250 nm, 250 nm to 260 nm, and 252 nm to 258 nm. In one embodiment, the one or more second light sources emit light with the same or different wavelength bandwidths (full wavelength bandwidth at half maximum intensity) at less than, equal to, or greater than one selected from the group: 1 nm, 2 nm, 3 nm, 4 nm, 5 nm, 10 nm, 15 nm, 20 nm, 25 nm, 30 nm, 40 nm, 50 nm, and 70 nm.

In one embodiment, the light emitting device comprises one or more visible light sources that may be used to indicate a status of the light emitting device and/or light source or to provide a visible representation of the angular light spread from the invisible UV-C light emitting downwards and/or upwards. In one embodiment, the angular full-width at half maximum luminous intensity of the one or more visible light sources in one or two orthogonal planes of light including the nadir (or orthogonal to the light emitting surface of the light emitting device) is within 5%, 10%, 15%, or 20% of the angular full-width at half maximum radiant intensity of the light from the one or more first light sources (such as UV-C light sources) in the same one or two orthogonal planes. In another embodiment, the angles at which the visible light intensity from the one or more visible light sources is less than 5% of the maximum visible light intensity in the one or two orthogonal planes of light including the nadir (or orthogonal to the light emitting surface of the light emitting device) are within 5%, 10%, 15%, or 20% of the angles at which the radiant intensity from the one or more first light sources (such as UV-C emitting light sources) is less than 5% of the maximum radiant light intensity in the same one or two orthogonal planes. In some embodiments, the angular bandwidth and/or angular cut-off angles of the visible light is matched to the UV-C light to visibly represent the illumination from the UV-C light, such that the visible light may be turned on (such as in a visible light representation mode) to illuminate the room to indicate which areas and/or surfaces would receive more or less UV-C light (including indicating shadow areas) without risk to UV-C exposure and/or additional UV-C exposure.

In one embodiment, the light emitting device comprises an indicator light source that emits visible light. In one embodiment, the light emitting device comprises one or more light sources that emits visible light to provide illumination in addition to one or more light sources emitting UV-C light. In one embodiment, a light emitting device comprises at least one broadband light source that emits light in a wavelength spectrum larger than 100 nanometers. In another embodiment, a light emitting device comprises at least one narrowband light source that emits light in a narrow bandwidth less than 100 nanometers. In another embodiment, a light emitting device comprises at least one broadband light source that emits light in a wavelength spectrum larger than 100 nanometers or at least one narrowband light source that emits light in a narrow bandwidth less than 100 nanometers. In one embodiment a light emitting device comprises at least one narrowband light source with a peak wavelength within a range selected from the group of 300 nm-350 nm, 350 nm-400 nm, 400 nm-450 nm, 450 nm-500 nm, 500 nm-550 nm, 550 nm-600 nm, 600 nm-650 nm, 650 nm-700 nm, 700 nm-750 nm, 750 nm-800 nm, and 800 nm-1200 nm. The light sources may be chosen to match the spectral qualities of red, green and blue such that collectively when used in a light emitting device, the color may be dialed in to achieve a desired color. In one embodiment, at least one light source is an LED package comprising a red, green, and blue LED capable of emitting light with a white color when each are emitting light. In another embodiment, the LED is a blue or ultraviolet LED combined with a phosphor. In another embodiment, a light emitting device comprises a light source with a first activating energy and a wavelength conversion material which converts a first portion of the first activating energy into a second wavelength different than the first. In another embodiment, the light emitting device comprises at least one wavelength conversion material selected from the group of a fluorophore, phosphor, a fluorescent dye, an inorganic phosphor, photonic bandgap material, a quantum dot material. In another embodiment, the light emitting device comprises white LED light sources. In another embodiment, the light sources comprise LEDs that are at least one selected from the group of: warm white, cool white, neutral white, daylight white, have a correlated color temperature between 2200 K and 2900 K, have a correlated color temperature between 2900 K and 3600 K, have a correlated color temperature between 3600 K and 4500 K, have a correlated color temperature between 4500 K and 4900 K, and have a correlated color temperature between 4900 K and 6600 K.

### Radiant Light Flux Output

In one embodiment, the one or more first light sources and/or one or more second light sources each emit light with a radiant flux (or the collective total flux) within the range of one or more selected from the group: 0.1 mW to 2 mW, 0.1 mW to 1.5 mW, 0.5 mW to 1.5 mW, 1 mW to 1.5 mW, 1 mW to 10 mW, 1 mW to 200 mW, 5 mW to 50 mW, 10 mW to 40 mW, 15 mW to 35 mW, 20 mW to 35 mW, 30 mW to 40 mW, greater than 10 mW, greater than 20 mW, greater than 30 mW, greater than 40 mW, greater than 50 mW, greater than 75 mW, greater than 100 mW, greater than 200 mW, less than 10 mW, less than 20 mW, less than 30 mW, less than 40 mW, less than 50 mW, less than 75 mW, less than 100 mW, and less than 200 mW. In one embodiment the radiant flux output for each (or collectively from all) of the one or more first light sources directed downward (and optionally for the one or more second light sources directed upwards) may be chosen based at least in part on the light emitting device mounting height, distance from the ceiling, angular light output profile, recommended exposure limitations based on the wavelength and light output at surfaces, and/or exposure sufficient for inactivation and/or reduction of a particular amount and/or type of pathogenic bioburden at surfaces and/or areas or volumes based on a desired duty cycle, on-time, or other light emitting mode.

In one embodiment, the total radiant flux output from the one or more first light sources directed downward divided by the total radiant flux output from the one or more second light sources directed upward when all light sources are emitting light at the maximum power allowed by the light emitting device is one or more selected from the group: greater than 1, less than 1, equal to 1, between 0.1 and 0.8, between 0.5 and 1, between 0.7 and 0.9, between 0.4 and 0.9, between 0.1 and 0.5, between 0.01 and 0.3, between 1 and 10, between 1 and 20, between 1 and 2, between 1 and 5, between 1 and 10, between 1 and 1.8, and between 1 and 1.5. In one embodiment, the ratio of the total maximum light output flux from the downward directed light to the upward directed light is chosen to optimize the inactivation and/or reduction in pathogenic bioburden for the upward and downward directed light while considering the safety due to exposure from the UV-C light in one or more light emitting modes of the light emitting device. In one embodiment, the above referenced ratio of the output flux is during an operational mode where both the one or more first light sources and the one or more second light sources are emitting light and the maximum light output is the maximum for a specific operational mode.

### Angular Light Modifier

In one embodiment, the one or more first light sources and/or one or more second light sources includes one or more angular light modifiers selected from the group of: reflector, light baffle, diffuser, and UV fused silica diffuser. In one embodiment, the angular light modifier increases or decreases the angular light output of the light emitting device. The first peak wavelength, first center wavelength, first average wavelength, first wavelength bandwidth, first light flux output, first angular light modifier, the second peak wavelength, second center wavelength, second average wavelength, second wavelength bandwidth, second light flux output, and/or second angular light modifier may be chosen for a particular efficacy in inactivation or reduction of one or more specific pathogenic bioburden and in consideration of safety of exposure to humans for a particular installation and/or light emitting mode.

### Air Flow

In one embodiment, the light emitting device comprises a fan or other air flow generation device (such as an electrostatic precipitator) that generates air flow across the first light flux from one or more first UV-C light sources and/or the second light flux from one or more second UV-C light sources and/or generates air flow through a particulate filter such as a HEPA filter. In one embodiment, the air flow generation device, such as a fan, generates air flow across the first light flux and/or second light flux from the one or more UV-C light sources, such as directing the air flow across the first flux from the first light sources emitting light with a peak wavelength of 222 nanometers (in the downward direction, for example) and/or the second light flux from the one or more second light sources emitting light with a peak wavelength of 254 nanometers (in the upward direction, for example). In one embodiment, the air flow generation device (optionally in conjunction with a ventilation system) maintains 0.35 to 8 air changes per hour which may be variable for residential or commercial installations.

### Sensors

In one embodiment, the light emitting device comprises one or more sensors configured to communicate data about the environment (environmental data) to a processor, such as remote processor or a processor in the UV-C light emitting device. In one embodiment, the light emitting device comprises one or more active or passive proximity sensors, occupancy sensors, or motion detectors positioned to detect motion beneath and/or above the light emitting device. In one embodiment, the one or more proximity sensors, occupancy sensors, or motion detectors are one or more selected from the group: infrared sensor, capacitive sensor, photoelectric sensor, doppler effect sensor, CCD sensor, CMOS sensor, ultrasonic sensor, microwave sensor, tomographic motion detector. In one embodiment, the light emitting device comprises two or more different active or passive proximity sensors, occupancy sensors, or motion detectors to increase accuracy. In one embodiment the light from the one or more first light sources directed downward is turned off or on when motion or occupancy is detected beneath the light emitting device. In another embodiment the light from the one or more second light sources directed upward is turned off or on when motion or occupancy is detected above the light emitting device. In one embodiment, the light emitting device comprises a UV imager that captures images representing relative values or calibrated absolute values of light reflected from the environment below (and optionally a second UV imager capturing from above) from which UV-C intensity and/or exposure can be estimated and/or calculated optionally with input from a user of the materials of the exposed surface (such as aluminum or wood) or the UV-C reflectances (at the wavelengths of the corresponding light sources) of the materials (which may be looked up in a data table by the software for the calculations). In this embodiment, a user may, for example, see on a display an image representing the specific UV-C exposure at the surfaces of the area (optionally using a false color map) which may indicate shadow areas, for example. In another embodiment, the light emitting device comprises a visible light imager which captures a visible light image of the environment, upon which an overlay of the UV-C exposure may be overlaid in a false-color map or other visible indication on the display.

In one embodiment, the light-emitting device includes an infrared photodetector, a phototransistor or an infrared (IR) receiver disposed to receive IR light. For example, in one embodiment, the light flux output from the one or more first light sources directed downward and/or the one or more second light sources directed upward is increased, decreased, changed, or turned off in response to information received from or through an infrared receiver from an infrared remote control.

In one embodiment, a UV-C sensitive spot meter or UV-C sensitive imager is used to evaluate the UV-C irradiation at a particular location/area in the environment receiving UV-C light from the light emitting device. In one embodiment, the radiant flux output from the one or more first light sources directing light downward and/or the radiant flux output from the one or more second light sources directing light upward is adjusted based on input information from a UV-C spot meter or a UV-C imager. In one embodiment, a handheld UV-C spot meter, fixed location UV-C spot meter or detector (such as a UV-C spot meter attached to arm that attached to the light emitting device that may be swung out to receive UV-C light from the light emitting device and later rotated back to the housing), or UV-C imaging device is used in a UV-C illumination system comprising a UV-C light emitting device as disclosed herein and the output from the UV-C spot meter is manually or automatically, input (such as by using IEEE 802.11, Bluetooth, serial USB or other communication protocol, for example) into software or an application on the light emitting device or in communication with the light emitting device, such that the radiant light flux output from the one or more first light sources and/or the radiant light flux output from the one or more second light sources may be evaluated based on lifetime information, or manually or automatically adjusted. This manual or automatic adjustment may achieve a target radiant light flux output at the UV-C radiant intensity evaluation location, achieve a target overall light flux output, or achieve a specific target light flux output at another location based on relative known or input parameters. In one embodiment, the radiant light flux from the one or more first light sources or the one or more second light sources may be evaluated using the spot meter or UV-C imager, such as for example, to facilitate achieving a desired output radiant intensity and/or desired exposure. In one embodiment the light emitting device comprises a user adjustable light output knob, dial, slide, or other variable adjustment mechanism that can be adjusted to increase and/or decrease the radiant light flux output from the one or more first light sources oriented downward and/or the one or more second light sources oriented upward to achieve a specific radiant light flux output from the one or more first light sources and/or the one or more second light sources in real-time while a user is viewing a display, dial, or indicator of the UV-C spot meter or UV-C imager. In one embodiment, the UV-C imager is a CCD imager with a UV-C bandpass filter (and/or short pass filter or other filter that absorbs and/or reflects greater than 80% of light with wavelengths longer than 280 nm and a phosphor conversion layer that down converts incident 222 nm light to light of a longer wavelength such that the CCD can detect the UV-C light. In one embodiment a method of adjusting the light output from a light emitting device comprising one or more first light sources oriented downward and/or one or more second light sources oriented upward comprises adjusting (manually and/or automatically) the radiant light flux output of the one or more first light sources oriented downward and/or the one or more second light sources oriented upward (optionally independently) based on information from a UV spot meter and/or a UV-C imager which may be portable, an attachment or accessory to a portable device such as a cellphone, in a fixed location (in the environment or on the light emitting device), or on an arm or extension of the light emitting device that may be translated and/or rotated into the UV-C light output from the light emitting device. In one embodiment, a system comprises one or more light emitting devices constructed to emit UV-C radiation, one or more processors, and network of external sensor stations, such as a Wireless Sensor Network (WSN).

### Temperature Sensor

In one embodiment the light emitting device comprises a temperature sensor or is in electrical or wireless communication with a temperature sensor. In one embodiment, a system comprising the light emitting device comprises one or more temperature sensors which may be located on the light emitting device, located remotely from the light emitting device, or located on a remote mobile device such as a portable phone, handheld device, or autonomous robot. In one embodiment, a system comprising the light emitting device comprises a non-transitory computer-readable storage medium comprising data representing a plurality of temperature ranges including at least a first temperature range and a second temperature range that may be one or more selected from the group: pre-installed, default, user adjustable, remotely adjustable by a user, adjustable remotely by a manufacturer or entity, updated by the user, updated by the manufacturer, and/or updated by a service provider such as a sanitation-as-a-service provider. In one embodiment, the first temperature range includes a range of temperatures selected from one or more of the group: below 32 degrees, between 32 and 40 degrees, between 40 and 50 degrees, between 50 and 60 degrees, between 60 and 70 degrees, between 70 and 80 degrees, between 80 and 90 degrees, between 90 and 100 degrees, between 100 and 110 degrees, between 110 and 120 degrees, between 120 and 130 degrees, between 130 and 140 degrees, greater than 50 degrees, greater than 60 degrees, greater than 70 degrees, greater than 80 degrees, greater than 90 degrees, greater than 100 degrees, and greater than 110 degrees Fahrenheit. In another embodiment, the second temperature range includes a range of temperatures different from the first range of temperatures selected from one or more of the group: below 32 degrees, between 32 and 40 degrees, between 40 and 50 degrees, between 50 and 60 degrees, between 60 and 70 degrees, between 70 and 80 degrees, between 80 and 90 degrees, between 90 and 100 degrees, between 100 and 110 degrees, between 110 and 120 degrees, between 120 and 130 degrees, between 130 and 140 degrees, greater than 50 degrees, greater than 60 degrees, greater than 70 degrees, greater than 80 degrees, greater than 90 degrees, greater than 100 degrees, and greater than 110 degrees Fahrenheit.

### Humidity Sensor

In one embodiment the light emitting device comprises a humidity sensor or is in electrical or wireless communication with a humidity sensor. In one embodiment, a system comprising the light emitting device comprises one or more humidity sensors which may be located on the light emitting device, located remotely from the light emitting device, or located on a remote mobile device such as a portable phone, handheld device, or autonomous robot. In one embodiment, a system comprising the light emitting device comprises a non-transitory computer-readable storage medium comprising data representing a plurality of humidity ranges including at least a first humidity range and a second humidity range that may be one or more selected from the group: pre-installed, default, user adjustable, remotely adjustable by a user, adjustable remotely by a manufacturer or entity, updated by the user, updated by the manufacturer, and/or updated by a service provider such as a sanitation-as-a-service provider. In one embodiment, the first humidity range includes a range of temperatures selected from one or more of the group: below 5%, below 10%, below 20%, below 30%, below 40%, below 50%, below 60%, below 70%, below 80%, between 10% and 20%, between 20% and 30%, between 30% and 40%, between 40% and 50%, between 50% and 60%, between 60% and 70%, between 70% and 80%, between 80% and 90%, between 90 and 100%, greater than 20%, greater than 30%, greater than 40%, greater than 50%, greater than 60%, greater than 70%, greater than 80%, and greater than 90% relative humidity. In another embodiment, the second humidity range includes a range of humidity different from the first range of humidity selected from one or more of the group: below 5%, below 10%, below 20%, below 30%, below 40%, below 50%, below 60%, below 70%, below 80%, below 90% between 10% and 20%, between 20% and 30%, between 30% and 40%, between 40% and 50%, between 50% and 60%, between 60% and 70%, between 70% and 80%, between 80% and 90%, between 90 and 100%, greater than 20%, greater than 30%, greater than 40%, greater than 50%, greater than 60%, greater than 70%, greater than 80%, and greater than 90% relative humidity.

### Controlling and Monitoring the Light Emitting Device

In one embodiment, control for the light emitting device is through a user interface at the light emitting device, remote from the light emitting device using a wired connection, or remote from the light emitting device using a wireless connection. In one embodiment, the light emitting device comprises a user interface for controlling the light emitting device at the light emitting device. In one embodiment, the user interface at the light emitting device comprises one or more user interface devices selected from the group: switch, dial, knob, button, pull cord/chain/string, touch sensitive switch or interface (such as a capacitive based touch-sensitive region of the device or a touchscreen, and microphone (for user commands, for example).

In another embodiment, the light emitting device comprises a wired control connection wherein one or more light output properties of the light emitting device is controlled using a wired connection to the light emitting device. In one embodiment, the control connection wired to the light emitting device comprises one or more remote operated wired control devices, such as a user interface (such as three flip switches) in a wall near a door, or a user interface on a wall (such as a light switch). In one embodiment, a system for controlling a light emitting device comprises a light emitting device and a control module comprising one or more user interfaces operatively configured to control the light output from the one or more first light sources and optionally, independently from the light output from the one or more second light sources of the emitting device.

In another embodiment, the light emitting device comprises a wireless control connection wherein one or more light output properties of the light emitting device are controlled using a wireless connection to the light emitting device. In one embodiment, the wireless control device wirelessly connected to the light emitting device comprises one or more remote operated wireless control devices, such as a wireless user interface device in a wall near a door, a wireless user interface remote from the light emitting device, or a portable device (such as controlling the light emitting device using an application on a wireless phone).

In one embodiment the light flux output of the one or more first light sources directed downward and/or the one or more second light sources directed upward (if provided) may be controlled or adjusted in a time period (optionally independently) using one or more light emitting modes selected from the group of: manual control, automatic control, programmed time control or schedule, duty cycle control, preset mode, another mode (such as manual mode) with control overridden based on input from a sensor (such as upward directed or downward directed motion sensor), test mode (such as visible light test mode that represents the UV-C light output), visible light illumination and UV-C light output mode, light flux output ratio for the one or more first light sources to the one or more second light sources, light source life maximizing mode that maximizes the lifetime of the one or more first light sources and/or one or more second light sources, maximum inactivation or reduction of pathogenic bioburden mode, optimized energy saving mode for a desired variable (such as exposure time, exposure intensity, or occupancy prediction/estimation, for example, such as mode uses the shortest exposure time for effective inactivation and/or reduction of pathogenic bioburden from light from the one or more first light sources and/or one or more second light sources to minimize electrical power consumed).

In one embodiment, the system comprising the UV-C light emitting device and a processor analyzes one or more environmental or other variables disclosed herein and optionally using machine learning or neural network generates an optimum duty cycle for inactivating and/or reducing pathogenic bioburden in the environment taking into account the height from the surface (such as the floor) of the environment of the UV-C light emitting device, the overall size/square footage of the environment, and the occupancy of the environment. In one embodiment, the system comprising the UV-C light emitting device and processor is a platform that allows a range of devices to be monitored, controlled, and or coordinated to ensure the safety of the inhabitants.

In one embodiment, the light emitting device comprises one or more microprocessors and at least one non-transitory computer-readable storage medium that collectively store the time duration (run time or counter) of emitting light of the one or more first light sources, and/or the one or more second light sources, and/or the light emitting device. In one embodiment, the light emitting device provides a visible (such as through a visible LED blinking or color change) indication, and/or audio indication, and/or visibly display indication through an application or software on a the light emitting device, portable device, remote server, remote computer, remote operating device, or remote building management computer system when the run time of the one or more first light sources, and/or the one or more second light sources, and/or the light emitting device reaches a time threshold. In one embodiment the time threshold is a threshold time less than one selected from the group of 40%, 30%, 20%, 15%, 10%, and 5% of the expected lifetime remaining for one or more of the first and/or second light sources. For example, in one embodiment, a red LED indicator emits red light downward when the duration of the 222 nm UV-C light source has reached a remaining lifetime of 20% of an expected lifetime of 3,000 hours. In one embodiment, the light emitting device comprises a button, a switch, dial, or a user input device of a system in communication with the light emitting device that resets the run time counter. In one embodiment, a side or the lower side of the light emitting device comprises one or more displays that indicate the number of hours the one or more first light sources and/or the one or more second light sources have been emitting light. In another embodiment an LED emits green, red, blue, white, or other color visible light when the light emitting device is emitting UV-C light.

### Control Hardware and Protocol

In one embodiment, one or more light emitting devices which may be positioned or installed in one or more locations and/or buildings and may be controlled (and/or the light output, light emitting mode, or other status information may be determined) in one or more light emitting modes using one or more communication methods (optionally in communication with a remote device, remote server, remote web server, remote processor, or website) such as a wired ethernet connection to the light emitting device, or wireless radio communication to the light emitting device (such as an IEEE 802.11 protocol (Wi Fi), Bluetooth® protocol, Z-wave protocol, or Zigbee protocol, for example).

In one embodiment, the light emitting device communicates with a second device used in a wired connection. In another embodiment, the connection between the light emitting device and the second device includes one or more of the following connections: serial, asynchronous serial, parallel, and USB. In one embodiment, the light emitting device communicates with a second device using one or more communication architectures, network protocols, data link layers, network layers, network layer management protocols, transport layers, session layers, and/or application layers.

In one embodiment, the light-emitting device has a radio frequency transmitter and a receiver that receives and transmits information. In a further embodiment, the light emitting device changes a property due to radio frequency communication with a device. In one embodiment, the radio frequency transmitter transmits and receives the frequency-hopping spread spectrum radio technology. In another embodiment, the light-emitting device includes a radio transmitter and receiver that receives and transmits radiation by Gaussian frequency-shift keying (GFSK). In another embodiment, the light-emitting device has a short wavelength radio transmission protocol, such as Bluetooth® protocol, radio frequency transmitter and receiver that receives and transmits information. In another embodiment, the light-emitting device includes an IEEE 802.11 compliant radio transmitter and receiver. In another embodiment, the light-emitting device includes an IEEE 802.15.4-2003, ZigBee® RF4CE, or ZigBee® compliant radio transmitter and receiver. For example, in one embodiment, the light emitting device receives information from a wireless router using an IEEE 802.11 protocol that directs the light emitting device to change the light emitting mode, provide an update on the status of the light emitting device, or directly change the light flux output from the one or more first light sources directed downward and/or the one or more second light sources directed upward.

In another embodiment, the light-emitting device includes a radio transceiver compliant to at least one communication standard for creating a wide area network (WAN) selected from the group of: iBurst™, Fast Low-latency Access with Seamless Handoff-Orthogonal Frequency Division Multiplexing (Flash-OFDM™), Wi-Fi: 802.11 standard, WiMAX: 802.16 standard, UMTS over W-CDMA, UMTS-TDD, EV-DO x1 Rev 0, Rev A, Rev B and x3 standards, HSPA D and U standards, RTT, GPRS, and EDGE. In another embodiment, the light-emitting device includes a radio transceiver compliant to at least one communication standard for creating a local area network (WLAN) selected from the group of: IEEE 802.11-2007, IEEE 802.11a, IEEE 802.11b, IEEE 802.11g, IEEE 802.11n, and amended IEEE 802.11-2007 standards or protocols. In a further embodiment, the light-emitting device includes a radio transceiver compliant to at least one communication standard for creating a personal area network (WPAN) selected from the group of: short wavelength wireless transmission protocol, such as Bluetooth® protocol (including standard protocol and low energy protocol), high level communication protocol such as ZigBee® Wireless USB, UWB, IPv6 over Low power Wireless Personal Area Networks, ONE-NET™, Z-Wave®, and EnOcean® standards. In another embodiment, the light emitting device includes a transceiver disposed to receive and transmit radio frequency information over a cellular phone connection protocol selected from the group of: CDMA, GSM, EDGE, 3G, UMTS, and SMS. In another embodiment, the light-emitting device includes 2 or more radio frequency transceivers configured to receive similar or different protocols, such as Bluetooth® protocol and an IEEE 802.11 protocol, for example.

In one embodiment, the light emitting device communicates with a building management or automation system (such as a computer network communicating using ASHRAE BACnet protocol, for example). In this embodiment, the light emitting device may be controlled and/or provide information to a building management system that can control the light emitting device according to one or more light emitting modes and/or instructions which can take into account information from other sensors in the system (such as motion detectors on light emitting devices just outside of a door to a room) with the light emitting device comprising one or more UV-C light sources detecting movement and instructing the second light emitting device to stop emitting UV-C light from the one or more first light sources and/or the one or more second light sources.

In one embodiment, the light emitting device is controlled to adjust the radiant light flux of the one or more first light sources directed downward and/or the one or more second light sources directed upward using software operating on a processor on the light emitting device and/or remote from the light emitting device based on one or more input selected from the group: information from one or more sensors, initial input from the manufacturer, subsequent updated information from the manufacturer (such as a firmware or data update), user input and selections, user selection of one or more light emitting modes and associated parameters, using one or more default light emitting modes and default parameters, input information or light emitting mode selection or parameters from a third party (such as a building manager), input instructions, sensor, or parameter information from a network comprising building management software and/or other UV-C light emitting devices or light fixtures.

### Standardization of Control

In one embodiment, a system for inactivating and/or reducing pathogenic bioburden comprises a set of standardized control and/or interface protocol for a plurality of UV-C light emitting devices that enables a processor to interact, and/or receive sensor or other data from a plurality of UV-C light emitting devices from different manufacturers (or the same manufacturer) and also control the plurality of UV-C light emitting devices independently to inactivate and/or reduce pathogenic bioburden in the environment. The standardization may be for a particular set or group of light emitting devices, air flow devices, sensors, humidifiers, compressors, robots which may be from different manufactures that may adopt usage of the standardized control and/or interface protocol. In one embodiment, the processor analyzes a metric for evaluating the exposure in an environment. In another embodiment, the metric comprises an irradiance exposure at a particular wavelength per unit airflow. In one embodiment, the exposure may be represented by millijoules per square centimeter or millijoules per square centimeter per cubic feet per minute at wavelength lambda, where the wavelength may be in a particular wavelength range or bin disclosed herein, or at a particular wavelength such as 222 nanometers or 254 nanometers.

### Algorithms and Machine Learning for Controlling the Light Emitting Device

In one embodiment, a system comprises one or more light emitting devices constructed to emit UV-C radiation and one or more processors adapted to perform one or more of the following functions (optionally using artificial intelligence learning techniques and/or neural network techniques and systems): to recognize the current state of the environment (and optionally create an environmental quality map) and adjust the light output or other output of the one or more light emitting devices constructed to emit UV-C radiation; collect and/or process sensor data; determine empirical or programmed data relationships; identify patterns from input data (such as sensor data or other data input to the processor); correlate relationships with input data, sensor data, and/or processed data; and build and/or analyze one or more environmental quality maps.

In one embodiment, the system comprises one or more processors configured to use one or more probabilistic models to perform nonlinear classification and regression or approximate a mapping from input space to output space using data from sensors (optionally historical or in real-time) and/or one or more variables disclosed herein to determine output that controls one or more light emitting devices or other devices disclosed herein (such as air flow devices, robots, etc.).

In one embodiment, the system comprises one or more processors configured to use one or more neural network types (single or multi-layer) selected from the group: feed forward network, radial based network, perceptron, multi-layer perception, convolutional neural networks, recurrent neural networks, and long short-term memory network and adjust the light flux output from one or more UV-C light emitting devices based on the analysis by the one or more processors using the one or more neural network types.

In one embodiment, the protocol for controlling the light emitting device provides suggestions to a user using a mobile application on how to adjust one or more variables for the light output of one or more of the light emitting devices to create a specific air quality map for one or more regions or spaces (such as a specific room, hallway, reception, interior area, etc.).

In one embodiment, the system advises the user (and/or the manufacturer of the light emitting device) or inhabitant how to optimize the environment (including one or more environmental metrics or parameters) based on a network of sensor data (such as real-time sensor data or sensor data delayed by less than 5 seconds, 10 seconds, 30 seconds, 1 hour, 2 hours, 4 hours, 8 hours, 12 hours, or 24 hours), via a Wireless Sensor Network (WSN), via autonomous (or programmed, or manually operated) mobile sensors such as on a robot or handheld device, and/or suspended or otherwise stationary sensors capable of real-time or delayed sensor data output.

In one embodiment, the system notifies (optionally through an application on a mobile device) one or more users (and/or the manufacturer of the light emitting device) of impending issues/warnings such as a high temperature (or a predictive high temperature) or gas levels outside of a normal and/or predetermined ranges. For example, in one embodiment, the system comprises one or more light emitting devices constructed to emit UV-C radiation, one or more sensors that detect humidity or temperature (or the system receives data from one or more sensors outside of the system but in communication with the system), a processor analyzing data from the one or more sensors determines that the environment (or a specific portion of the environment) of the light emitting device is within a first temperature range, first humidity range, first bacteria range (such as colony-forming unit per gram, CFU/g range), and/or first irradiance range (such as UV-C irradiance) and the processor directs one or more of the following actions: increase or decrease the light flux output from one or more light emitting devices; increase or decrease the air flow from one or more air handlers, fans, or HVAC systems (or an air flow device such as a fan within the light emitting device) to increase or decrease the air flow, increase or decrease the air temperature of the air flow to the environment, condition the environment, reduce or increase the humidity of the air flowing to the environment, such as, for example, to lower the humidity of the environment to an optimal or predetermined range for improved comfort and/or inactivating and/or reducing pathogenic bioburden.

In one embodiment, the system comprises one or more light emitting devices constructed to emit UV-C radiation, one or more sensors (or the system receives data from one or more sensors remote from the UV-C light emitting device but in communication with the system), a processor analyzing data from the one or more sensors to determine patterns, correlations, or input and output relationships, and the system sends the output from the analyzed data to a user and/or the manufacturer of the light emitting device (optionally via an application on a mobile device) and/or controls (or directs) one or more aspects of the environment (such as fan speed, cooling compressor on or off, duration of fan or compressor on-time, air circulation, or cubic feet per minute air flow, for example) and/or light flux output (total light flux output, light flux duration, and/or light flux output frequency) from one or more light emitting devices constructed to emit UV-C radiation (such as the radiant flux and/or duration UV-C light output upward and/or downward from one or more light sources, for example) in order to optimize the environment or change one or more environmental parameters or metrics based on a predetermined, user controlled, or programmed desired range or value to increase comfort, inactivate and/or reduce pathogenic bioburden, or increase safety, for example. In one embodiment, a system for inactivating and/or reducing pathogenic bioburden comprises on or more processors that analyze data from a plurality of sensors and directly modify the environment (such as UV light irradiance from one or more fixtures emitting light up or down, air flow, humidity, etc.) and/or sends an indication to a processor that is in communication with the system and/or a portable device (such as an application) to modify the environment.

In one embodiment, the system comprises one or more light emitting devices constructed to emit UV-C radiation into the environment and a system on chip, “SoC”, computer design that enables the addition of one or more sensors to work together to reduce to inactivate and/or reduce pathogenic bioburden in the environment. In one embodiment, the SoC is capable of having additional sensors customized and added based on environmental factors that are specific to a given environment, such as humidity sensors in a bathroom environment, for example. In one embodiment, the light apparatus can control one or more UV-C light emitting modules (comprising one or more light sources) on one or more UV-C light emitting devices at a given time which can have alternated (or individually controlled) irradiation times, durations, and/or on-time frequencies to enhance the UV disinfection process based on environmental variables and/or user input parameters.

In one embodiment, the SoC data collection on UV-C light emitting devices or modules (and/or one or more light sources) light flux output acts as a failsafe for light emitting device, module, or light source failure and can adjust disinfection cycle parameters based on efficacy yield curve differentiation due to bulb or light source lifespan performance degradation. In one embodiment, the SoC sensor data is in a standardized format such that the device may be classified as an Internet of Things, “IoT”, device that can send data to the cloud or remote servers on network. In one embodiment, the data from one or more sensors on one or more UV-C light emitting devices or sensors in a system comprising one or more UV-C light emitting devices is aggregated and the data can be used by one or more different UV-C light emitting devices (or other devices such as disclosed herein) or in one or more systems comprising one or more different UV-C light emitting devices to optimize, inactivate and/or reduce pathogenic bioburden in the environment.

In one embodiment, one or more UV-C light emitting devices (or a system comprising one or more UV-C light emitting devices and a processor) is programmatically configured to use or take into account updated schedules through asynchronous internet connectivity to change disinfection cycles (or cycles to inactivate and/or reduce pathogenic bioburden in the environment) which may include, for example changing the UV-C light flux output, temperature, humidity, or other variable in the environment disclosed herein to optimize the disinfection cycles (or cycles to inactivate and/or reduce pathogenic bioburden in the environment).

For example, in one embodiment, a UV-C light emitting device (or system comprising the light emitting device) comprises a LiDAR sensor and a machine vision equipped camera (optionally embedded in the UV-C light emitting device) to determine areas of disinfection where an autonomous robotic vehicle creates a cleaning routine where efficacy may otherwise be compromised. In another embodiment, a UV-C light emitting device (or system comprising the light emitting device) comprises a LiDAR sensor, humidity sensor, and particulate sensor, and the system or UV-C light emitting device maps the distance to surfaces and atmospheric composition of the environment to determine optimal baseline disinfection exposure (light flux output, duration, and frequency) and frequencies of exposure, which may be further modified as particulate composition in the environment change over time to inactivate and/or reduce pathogenic bioburden in the environment. In one embodiment, a plurality of UV-C light emitting devices UV lights are connected (internal to the light emitting device, wirelessly, and/or wired) to a processor configured to communicate with a calendar software application of a user connected through an application through the Internet or other wireless communication protocols (e.g., Bluetooth, WiFi) whereas UV-C light exposure (light flux output and duration) and frequencies of exposure are adjusted based on user scheduling and/or movement activity.

### Air Quality or System Map

In one embodiment, the light flux output from one or more light emitting devices emitting UV-C light is controlled based on sensor data from multiple locations within a room or environment and/or multiple rooms or defined environments, which may be visualized as an air quality map or system map on a remote computer interface including a portable device. In one embodiment, the air quality map or system map displays one or more of the following measured, calculated, and/or estimated variables which may be optionally selectable past variables, current variables, future estimated variables, or scheduled variables and also which may be absolute or relative values in a spatial location map (and/or optionally metrics for the system and/or targets set for the system to adjust to achieve): air flow rate, number of occupants, light flux output upward, light flux output downward, radiant intensity on surface or region, lamp life (such as hours on or hours remaining), sanitation status, mobile disinfection robot location, location of UV-C light emitting devices, light flux output range of light emitting devices (upward or downward, for example), suspended particulate count, temperature, humidity, carbon dioxide content (such as parts per million or %), oxygen content (such as parts per million or %), nitrogen content (such as parts per million or %), contaminant gas or material content (such as parts per million or %), UV-C radiant intensity as a particular height in the environment, sound level, system efficacy level (such as a measured or estimated) reduction in colony-forming unit (CFU) (per milliliter) reduction in % of bioburden, absolute value (measured or estimated) of colony-forming unit (CFU) (per milliliter).

### Autonomous Robot

In one embodiment, the system for inactivating and/or reducing pathogenic bioburden comprises one or more autonomous or semi-autonomous robots. In one embodiment, the robot is one or more selected from the group: humanoid, drone, wheeled machine, tracked machine, mobile robot, industrial robot, service robot, modular robot, collaborative robot, mobile UV-C disinfection robot, and mobile UV-C irradiance sensing robot. In one embodiment, the robot moves to identified, scheduled, and/or adaptively learned locations in the environment and exposes directly from one or more UV-C light sources on a body surface of the robot one or more surfaces in the environment with UV-C radiation, or exposes from one or more UV-C light sources on an extended arm or structure extending from the body of the robot. In another embodiment, the robot moves to identified, scheduled, and/or adaptively learned locations in the environment and senses directly from a body surface of the robot the UV-C irradiance or senses the UV-C irradiance from a sensor on an extended arm or structure extending from the body of the robot. For example, in one embodiment, the robot is scheduled to expose an operating room table or bathroom stall area once a day at 4 AM (such as may be programmed using software on a mobile device), after an occupancy sensor detected that someone left the area more than 5 minutes ago, and/or based on an occupancy sensor detected heavy bathroom use during the past hour and no one is currently present in the bathroom. In one embodiment, sensors on the robot detect low UV-C irradiance from one or more light emitting devices and provides supplemental UV-C radiation or directs the one or more light emitting devices to stop emitting light upon the surface and the robot provides full exposure needed to inactivate and/or reduce pathogenic bioburden down to an acceptable level or concentration. In one embodiment, the robot provides one or more UV-C sensors and comprises one or more UV-C light sources positioned to emit UV-C light into the environment. In another embodiment, the robot comprises an air flow device, such as a fan, to circulate air in the environment to increase the air flow across an installed UV-C light emitting device or across a volume exposed by UV-C light emitted by the robot (which may be exposing light into the environment or may comprise an internal chamber or reactor that exposes air flowing through the chamber or reactor to the UV-C light without substantially exposing the environment external to the robot directly with the UV-C light. In one embodiment, the robot comprises an articulated arm and one or more UV-C light sources and/or one or more sensors (such as UV-C light detectors) that can change the orientation and/or position of the one or more UV-C light sources and/or one or more sensors to direct the UV-C light flux in a specific direction or on a specific surface of the environment or sense light or other variables from a specific location and/or orientation in the environment, respectively, in a sequential (serial) or simultaneous manner. For example, the arm can position a UV-C light source to emit light upward while simultaneously sensing the UV-C light reflected from the ceiling. In another example, the arm can position and orient a UV-C light detector in an environment with a UV-C light emitting device (such as directly beneath) to evaluate the irradiance in the environment (at a particular location or across the environment) from that particular UV-C light emitting device only and repeating for other UV-C light emitting devices in the environment to map out the irradiance from each UV-C light emitting device.

In one embodiment, the environment exposed with UV-C light from the light emitting device comprises a mirror (such as in a bathroom) or UV-C reflective surface (such as a high reflectance white paint or material with a UV-C reflectance greater than 70%, aluminum, and/or expanded polytetrafluoroethylene) and the UV-C irradiance from one or more UV-C light emitting devices is evaluated using a sensor and/or calculated taking into account UV-C light reflected (with some absorption measured, estimate, or calculate)from the mirror or UV-C reflective surface.

### Other Components of the Light Emitting Device

In one embodiment, the light emitting device comprises one or more power supplies, AC/DC converters, control circuits, communication modules, radio transceiver, wiring, housing, connectors, switches, dials, buttons, light source access panel, or other devices known to be used in UV-C light irradiation devices and/or visible light illumination devices irradiating and/or illuminating a room or area.

In one embodiment, the light emitting device is powered by an electrical signal selected from the group of 12V DC, 12V AC, 110-277V AC, 220-240V AC, switchable power supply, 28V DC power supply, AC power supply, DC power supply, and 3V DC power supply. In another embodiment, the light emitting device has a backup battery based power supply.

### Light Source Access

In one embodiment, the light emitting device comprises a housing comprising the one or more first light sources and/or the one or more second light sources. In one embodiment, the housing has an access panel, access door, access flap, or access opening to permit access from beneath the light emitting device (such as an overhead light fixture) when installed such that the light source may be changed. In one embodiment, the one or more first light sources and/or the one or more second light sources are bulbs or field replaceable light sources. In this embodiment, the one or more power supplies may be optionally replaceable.

### Other Components of the Light Emitting Device

In one embodiment, the light emitting device or system comprising a light emitting device comprises one or more selected from the group: power supply, driver, battery, photovoltaic cell, photosensor (for detecting ambient light levels or change in light output or color from LEDs over time, for example), occupancy sensor, infrared light sensor, microphone, speaker, alarm, smoke detector, carbon monoxide detector, radio transceiver, microcontroller, non-transitory computer-readable storage medium, and communication interface port (such an RJ11, RJ45, USB, mini-USB, or other electronic device communication transfer port, for example). In one embodiment, the light emitting device comprises an occupancy sensor or door sensor (such as a magnetic door sensor) or is electrically or communicatively coupled to the sensor such that the light emitting device stops or starts emitting UV-C light when movement is detected, the door is opened, or occupancy is otherwise detected. In one embodiment, the portable device and/or vehicle comprise one or more processors (such as microprocessors) operatively configured to execute one or more algorithms, analyze information, communicate information, and/or execute one or more operational or light emitting modes for the light emitting device or system comprising the light emitting device. One or more algorithms disclosed herein may be executed on one or more processors of the light emitting device, portable device, or a remote device (such as a remote server). In one embodiment, the light emitting device, portable device, or remote device comprises software or software components executing one or more algorithms. The software and/or data may be stored on one or more non-transitory computer-readable storage media. The software may be the operating system or any installed software or applications, or software, applications, or algorithms stored on a non-transitory computer-readable storage medium of the light emitting device, portable device, and/or remote device.

### Light Emitting Device Type and Location

In one embodiment, the UV-C light emitting device is a light fixture or replacement bulb. In one embodiment, the light emitting device is a light fixture, can light, troffer light, cove light, recessed light, torch lamp, floor lamp, chandelier, surface mounted light, pendant light, sconce, track light, under-cabinet light, emergency light, wall-socket light, exit light, high bay light, low bay light, strip light, building light, outdoor light, accent light, flood light, wall-washer light, wall light, ceiling light, ceiling fan light, car light, outdoor flood light, or vehicle light. In one embodiment, the light emitting device is used in one or more selected from the group: an educational facility, hotel, restaurant, emergency room, doctor or dentist office, department of motor vehicle location, government office or public location, airport, dock, healthcare facility, retail location, workout facility or gym, gym shower, restroom or bathroom, meat-packing facility, poultry packing facility, food processing facility, theater, transit or transportation facility (such as a train or commuter rail or subway facility and/or their indoor or outdoor platforms) and office.

In one embodiment, the light emitting device or a system comprising the light emitting device comprises one or more selected from the group: mounts, accessories, fasteners, other components such as a stem kit, swivel ball hanger, T-bar box hanger for 2 foot by 2 foot mounting, and grid clips (such as grid clips for T-bars or suspended ceiling components) to enable the light emitting device to be attached to a common light fixture installation location, junction box, downlight light fixture can, box mounting clip.

### Shape of Light Emitting Device

In one embodiment, the UV-C light emitting device is substantially planar in a plane substantially orthogonal to the optical axis (or nadir of the light emitting device) of the one or more first light sources oriented downward. In one embodiment, the light emitting device is substantially linear (with a dimension in one first direction orthogonal to the optical axis (or nadir of the light emitting device) of the one or more first light sources oriented downward more than 3 times the dimension of the light emitting device in a second direction orthogonal to the first direction and the optical axis (or nadir of the light emitting device) of the one or more first light sources oriented downward. In one embodiment, the light emitting device is hemispherical or protrudes outward in a direction parallel to the optical axis (or nadir of the light emitting device) of the one or more first light sources oriented downward. In one embodiment, the light emitting device is in the shape of a rectangular cuboid, an apex-truncated square pyramid, or a prismatoid.

### Waterproof

In one embodiment, the light source and electrical components are substantially sealed by at least one of an epoxy, resin, rubber, silicone, or polymer such that the electrical components are waterproof to a depth selected from the group of 5 feet, 10 feet, 20 feet, 30 feet, 50 feet, 100 feet, and 200 feet. This can be useful, for example, in facilities with large numbers of people with surfaces that must be cleaned regularly or in environments where the light emitting device (such as a ceiling mounted light fixture) may be exposed to water, moisture, or high humidity, such as a gym shower, meat-packing facility, poultry packing facility, food processing facility, or outdoor train platform, for example. In another embodiment, the light emitting device components satisfy the United Laboratories UYMR2 standards for components and fittings intended for use in electric signs and accessories. In another embodiment, the light emitting device continues to operate after a 12 hour continuous salt spray test. In another embodiment, the light emitting device continues to operate after a 24 hour continuous salt spray test. In one embodiment, the light emitting device continues to operate after a 48 hour continuous salt spray test. In one embodiment, the light emitting device continues to operate after a 60 hour saltwater soak test. In one embodiment, the light emitting device continues to operate after a 120 hour saltwater soak test. In another embodiment, the light emitting device continues to operate after a 240 hour saltwater soak test.

The following are more detailed descriptions of various embodiments illustrated in the Figures.

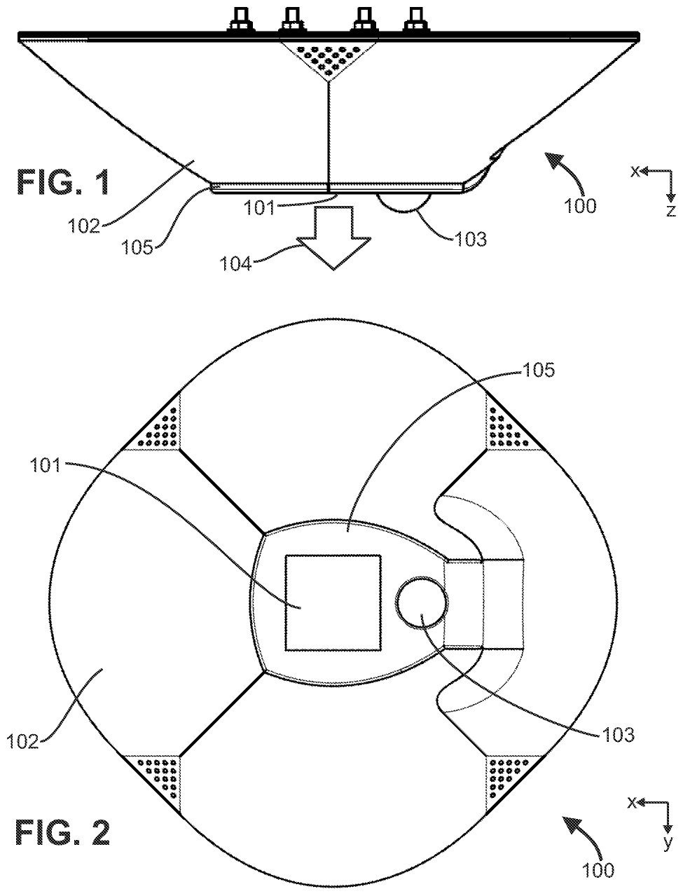

FIG. 1 is a side view of a light emitting device 100 comprising a UV-C light source 101 comprising one or more first light sources emitting UV-C light 104 downward when the light emitting device is mounted to a ceiling. The light emitting device 100 further comprises an occupancy sensor 103, a housing 102, and an access door 105 to enable access to the UV-C light source 101 to enable user replacement of the UV-C light source 101 from beneath the light emitting device 100. In one embodiment, the one or more first light sources emit 222 nm light.

FIG. 2 is a bottom view of the light emitting device 100 of FIG. 1.

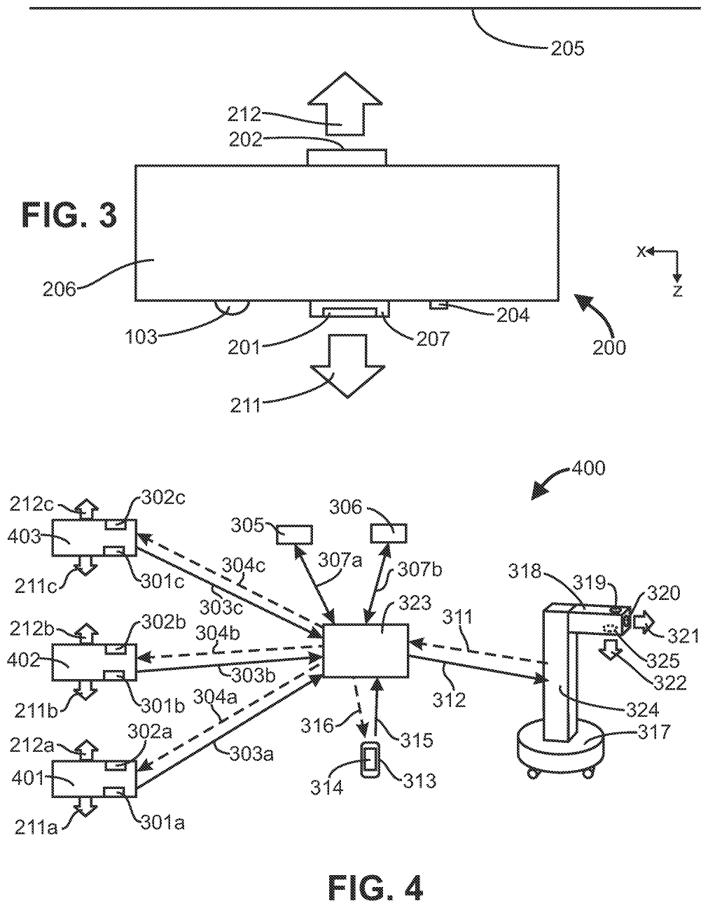

FIG. 3 is a side view of a light emitting device 200 comprising a first UV-C light source 201 comprising one or more first light sources emitting UV-C light 211 downward when the light emitting device 200 is suspended from a ceiling 205 using cables or chain, for example (not shown). The light emitting device 200 also comprises a second UV-C light source 202 comprising one or more second light sources emitting UV-C light 212 upward toward the ceiling 205. The light emitting device 200 further comprises an occupancy sensor 103, a second sensor 204, a housing 206, and an access door 207 to enable access to the first UV-C light source 201 to enable user replacement of the first UV-C light source 201 from beneath the light emitting device 200. In one embodiment, the one or more first light sources emits light with a peak wavelength of 222 nm light and the one or more second light sources emits light with a peak wavelength of 254 nm.

FIG. 4 is a schematic view of a system 400 for inactivating and/or reducing pathogenic bioburden in the environment comprising a first UV-C light emitting device 401, second UV-C light emitting device 402, and third UV-C light emitting device 403, emitting UV-C light downward (211a, 211b, 211c, respectively) emitting UV-C light upward (212a, 212b, 212c, respectively), comprising a first sensor (301a, 301b, 301c, respectively) on the lower side of the side of the UV-C light emitting device, comprising a second sensor (302a, 302b, 302c, respectively) on the upper side of the side of the UV-C light emitting device, configured to receive data (which may be instructions) (304a, 304b, 304c, respectively) from a processor 323 remote from the light emitting device (such as remote “cloud” server or a remote server controlled by a light emitting device manufacturer, or a remote server of a company providing remote disinfection service or inactivating and/or reduced pathogenic bioburden service, or server in a remote area of a building), configured to transmit data (303a, 303b, 303c, respectively) which may include sensor data from the first sensor 301 and/or the second sensor 302 to the processor 323 remote from the light emitting devices (401, 402, 403). The system 400 further comprises a remote sensor 305 remote from the UV-C light emitting devices (401, 402, 403) and remote from the processor 323 configured to transmit and/or receive data 307a from the processor 323. The system 400 further comprises a remote sensor 306 remote from the UV-C light emitting devices (401, 402, 403) and remote from the processor 323 configured to transmit and/or receive data 307ba from the processor 323. The system further comprises a robot 317 comprising a first UV-C light source 320 on an arm 318 extended horizontally from the body 324 of the robot 317 positioned to emit first UV-C light 321 with a directional component in a horizontal direction. The robot 317 further comprises a second UV-C light source 325 positioned on the underside of the arm 318 extended horizontally from the body 324 of the robot 317 positioned to emit second UV-C light 322 with a directional component in a downward direction. The robot further comprises a sensor 319 (such as a UV-light detector) on the upper side of the arm 318. In another embodiment, the robot comprises a third UV-C light source positioned on the top side of the arm positioned to emit UV-C light with a directional component in the upward direction (not shown). The system 400 further comprises a mobile device 313 (such as a cellphone) comprising a display with user interface 314 configured to transmit data 315 to the server 323 remote from the mobile device 313 and receive data 316 from the server 323. In one embodiment, the processor 323 receives data from one or more sensors (301a, 301b, 301c, 302a, 302b, 302c, 305, 306, 319), user input from the mobile device 313, and processes the data using at least one of a machine learning method, neural network analysis, user programmed schedule, variable or parameters to control one or more of the irradiance, duration of irradiance, and/or frequency of irradiance of one or more light flux output (211a, 211b, 211c, 212a, 212b, 212c, 322, and 323) to inactivate and/or reduce pathogenic bioburden in the environment.

### Equivalents

Those skilled in the art will recognize or be able to ascertain using no more than routine experimentation, numerous equivalents to the specific procedures described herein. Such equivalents are considered to be within the scope of the invention. Various substitutions, alterations, and modifications may be made to the invention without departing from the spirit and scope of the invention. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the invention and embodiments thereof. This application is intended to cover any adaptations or variations of the specific embodiments discussed herein. Therefore, it is intended that this disclosure be limited only by the claims and the equivalents thereof.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein. Unless indicated to the contrary, all tests and properties are measured at an ambient temperature of 25 degrees Celsius or the environmental temperature within or near the device when powered on (when indicated) under constant ambient room temperature of 25 degrees Celsius.

What is claimed is:

1. A system for reducing pathogenic bioburden in an environment comprising:
a light emitting device comprising one or more light sources emitting light flux with a wavelength within a range of 100 nanometers to 280 nanometers;
two or more sensors, each sensor generating environmental data; and
a processor communicatively coupled to the two or more sensors and the light emitting device, the processor performing an analysis on the environmental data from each sensor of the two or more sensors and adjusting the light flux emitted from the light emitting device based at least in part on the environmental data from the two or more sensors.

2. The system of claim 1 wherein the one or more light sources comprise a first light source with a first peak wavelength between 220 nanometers and 225 nanometers.

3. The system of claim 2 wherein the one or more light sources further comprises a second light source with a second peak wavelength at 254 nanometers.

4. The system of claim 3 wherein the first light source is positioned in the light emitting device to emit light downward and the second light source is positioned to emit light upwards when the light emitting device is suspended in a room or the environment.

5. The system of claim 1 wherein the two or more sensors include at least one temperature sensor generating temperature data of the environment and at least one humidity sensor generating humidity data of the environment.

6. The system of claim 5 wherein the light flux emitted from the light emitting device is adjusted to inactivate and/or reduce pathogenic bioburden in the environment at least in part on a temperature and a humidity of the environment sensed by the two or more sensors.

7. The system of claim 6 wherein the analysis includes a neural network analysis on at least the environmental data from the two or more sensors.

8. The system of claim 6 wherein the two or more sensors include at least one suspended particulate sensor generating particulate concentration data for the environment, and the analysis is based at least on the temperature, the humidity, and an estimated air flow rate determined at least in part by the at least one suspended particulate sensor.

9. The system of claim 6 wherein the two or more sensors include at least one motion sensor or occupancy sensor providing data for the environment, wherein the light flux emitted from the light emitting device is adjusted to inactivate and/or reduce pathogenic bioburden in the environment based at least on temperature, humidity, and occupancy of the environment sensed by the two or more sensors.

10. The system of claim 1 wherein adjusting the light flux includes at least two adjustments selected from a group: total light flux output, duration of light flux output, and frequency of light flux output.

11. The system of claim 1 further comprising an air flow generation device wherein air flow from the air flow generation device is adjusted based at least in part on the environmental data from the two or more sensors.

12. The system of claim 1 wherein the two or more sensors include at least one at least one spot meter sensitive to light with a wavelength within the range of 100 nanometers to 280 nanometers or an imager sensitive to light with a wavelength within the range of 100 nanometers to 280 nanometers.

13. The system of claim 1 further comprising a plurality of light emitting devices, each comprising one or more light sources emitting light flux with a wavelength within a range of 100 nanometers to 280 nanometers, wherein the light emitting device and the plurality of light emitting devices are located within a building and the processor is remote from the building.

14. A system for monitoring and reducing pathogenic bioburden in an environment comprising:
one or more light emitting devices, each comprising one or more light sources emitting light flux with a wavelength within a range of 100 nanometers to 280 nanometers;
two or more sensors including at least a temperature sensor and a humidity sensor, each sensor generating environmental data; and
a processor communicatively coupled to the two or more sensors and the one or more light emitting devices, the processor monitoring the environmental data from the two or more sensors, performing an analysis on the environmental data from the two or more sensors, and adjusting the light flux emitted from the one or more light emitting devices based at least in part on the environmental data from the two or more sensor.

15. The system of claim 14 wherein the one or more light emitting devices are located within a building and the processor is remote from the building.

16. The system of claim 14 wherein the two or more sensors include at least one motion sensor or occupancy sensor providing data for the environment, wherein the light flux emitted from the one or more light emitting devices is adjusted based at least on a temperature, a humidity, and an occupancy of the environment sensed by the two or more sensors to inactivate and/or reduce pathogenic bioburden in the environment.

17. The system of claim 14 wherein adjusting the light flux includes at least two adjustments selected from a group: total light flux output, duration of light flux output, and frequency of light flux output.

18. A method of reducing pathogenic bioburden in an environment, the method comprising:
providing one or more light emitting devices, each comprising one or more light sources emitting light flux with a wavelength within a range of 100 nanometers to 280 nanometers;
reading data for the environment from two or more sensors; and
analyzing the data for the environment from the two or more sensors using a processor and adjusting the light flux emitted from the one or more light emitting devices based at least in part on the data from each sensor using a machine learning or neural network technique that inactivates and/or reduces of pathogenic bioburden in the environment.

19. The method of claim 18 wherein the two or more sensors include at least one temperature sensor remote from the one or more light emitting devices generating temperature data of the environment and at least one humidity sensor remote from the one or more light emitting devices generating humidity data of the environment.

20. The method of claim 18 wherein the one or more light emitting devices and the two or more sensors are located within a building and the processor is remote from the building.
